(19) United States
(12) Patent Application Publication
(10) Pub. No.: US 2018/0256147 A1
(43) Pub. Date: Sep. 13, 2018

(54) BONE SUTURE ASSEMBLY

(71) Applicant: MININVASIVE LTD., Magal (IL)

(72) Inventors: Dror ROSNER, Holon (IL); Ronen RAZ, Magal (IL); Arnon MOSHAIUF, Atlit (IL)

(73) Assignee: MININVASIVE LTD., Magal (IL)

(21) Appl. No.: 15/451,733

(22) Filed: Mar. 7, 2017

(51) Int. Cl. A61B 17/04

(52) U.S. Cl. CPC A61B 17/0401; A61B 17/0487; A61B 2017/0496; A61B 2017/0417; A61B 2017/0408

(57) ABSTRACT

A bone suture assembly including a flexible sleeve, at least one lockable suture associated with the flexible sleeve and having a series of mutually spaced suture engagement elements extending along at least a portion thereof and at least one suture locking element defining a loop which can be lockably tightened in locking engagement with a corresponding one of the at least one lockable suture by application of tension to the at least one suture locking element, without requiring knotting thereof.

### REFERENCE TO RELATED APPLICATIONS

Reference is made to the following PCT Patent Applications and U.S. Provisional Applications which are believed to be related to the present application, the contents of which are hereby incorporated by reference herein:

Published PCT Patent Application No. WO 2016/038614, entitled “Padded Transosseous Suture” and published Mar. 17, 2016:

Published PCT Patent Application No. WO 2014/147619, entitled “Arthroscopic Surgical Device” and published Sep. 9, 2014:

Published PCT Patent Application No. WO 2013/102909, entitled “Arthroscopic Surgical Device” and published Jul. 11, 2013;

Published PCT Patent Application No. WO 2013/027209, entitled “Arthroscopic Surgical Device” and published Feb. 28, 2013;

Published PCT Patent Application No. WO 2013/027210, entitled “Circular Bone Tunneling Device Employing a Stabilizing Element” and published Feb. 28, 2013;

Published PCT Patent Application No. WO 2012/007941, entitled “Circular Bone Tunneling Device” and published Jul. 11, 2011;

U.S. Provisional Patent Application Ser. No. 61/802,958, entitled “Arthroscopic Surgical Device” and filed Mar. 18, 2013;

U.S. Provisional Patent Application Ser. No. 61/887,561, entitled “Arthroscopic Surgical Device” and filed Oct. 7, 2013;

U.S. Provisional Patent Application Ser. No. 61/636,751, entitled “Circular Bone Tunneling Device Employing a Stabilizing Element” and filed Apr. 23, 2012:

U.S. Provisional Patent Application Ser. No. 61/526,717, entitled “Circular Bone Tunneling Device” and filed Aug. 24, 2011;

U.S. Provisional Patent Application Ser. No. 61/714,813, entitled “Arthroscopic Surgical Device” and filed Oct. 17, 2012;

U.S. Provisional Patent Application Ser. No. 61/584,267, entitled “Circular Bone Tunneling Device” and filed Jan. 8, 2012;

U.S. Provisional Patent Application Ser. No. 62/273,632, entitled “Arthroscopic Surgical Device” and filed Dec. 31, 2015; and

PCT Patent Application PCT/IL2015/050978, entitled “Arthroscopic Surgical Device” and filed Sep. 24, 2015.

### FIELD OF THE INVENTION

The present invention relates generally to sutures, suture assemblies and suturing techniques which are related to bones.

### BACKGROUND OF THE INVENTION

Various types of sutures, suture assemblies and suturing techniques are known.

### SUMMARY OF THE INVENTION

The present invention seeks to provide improved suture assemblies and suture techniques particularly suitable for transosseous type suturing.

There is thus provided in accordance with a preferred embodiment of the present invention a bone suture assembly including a flexible sleeve, at least one lockable suture associated with the flexible sleeve and having a series of mutually spaced suture engagement elements extending along at least a portion thereof and at least one suture locking element defining a loop which can be lockably tightened in locking engagement with a corresponding one of the at least one lockable suture by application of tension to the at least one suture locking element, without requiring knotting thereof.

In accordance with a preferred embodiment of the present invention the at least one suture locking element includes an elongate flexible element having first and second ends. Additionally, the at least one suture locking element is configured to be lockably tightened by pulling on the first end of the elongate flexible element.

Preferably, the at least suture one locking element defines a loop which can be lockably tightened in locking engagement with a corresponding one of the at least one lockable suture at a location therealong intermediate two of the series of mutually spaced suture engagement elements.

In accordance with a preferred embodiment of the present invention the flexible sleeve is a generally cylindrical sleeve formed of suture material. Additionally or alternatively, the at least one suture locking element is formed of suture material.

In accordance with a preferred embodiment of the present invention the flexible sleeve is braided from surgical suture material. Additionally or alternatively, the flexible sleeve includes a compressible elongate cylindrical portion, which is integrally formed at an end thereof with a compressible ring-shaped collar portion.

Preferably, the at least one suture locking element includes first and second suture locking loop elements, which extend through an interior of the flexible sleeve and are attached thereto.

In accordance with a preferred embodiment of the present invention, each of the at least one suture locking element includes a length of suture material having a slip noose-like end defining the loop by winding an end portion of the length of suture material in a coil over an intermediate portion of the length of suture material. Additionally, a portion of the length of suture material adjacent a free end thereof extends outwardly through a portion of the flexible sleeve for anchoring the slip noose-like end to the flexible sleeve.

Preferably, at least one winding of the coil extends through a portion of the flexible sleeve. In accordance with a preferred embodiment of the present invention, operation of the slip noose-like end is such that the flexible loop can be readily tightened and locked in a tightened operative orientation by pulling on the length of suture material in a given direction relative to the coil and the flexible sleeve, whereby the slip noose-like end locks the length of suture material against retraction and consequent loosening of the locking engagement of the loop with the lockable suture without requiring knotting.

In accordance with a preferred embodiment of the present invention the series of mutually spaced suture engagement elements are in fixed positions along each of the at least one lockable suture.

Preferably, the bone suture assembly also includes at least one pulling suture looped through flexible sleeve and suitable for pulling the flexible sleeve into a transosseous channel in a bone.

There is also provided in accordance with a preferred embodiment of the present invention a method of retaining a suture onto a bone without requiring knotting including providing a bone suture assembly including a flexible sleeve, at least one lockable suture, extending through the flexible sleeve and having a series of mutually spaced suture engagement elements extending along at least a portion thereof, and at least one suture locking element defining a loop, inserting the flexible sleeve into a channel extending through the bone and lockably tightening the at least one locking element in locking engagement with the at least one lockable suture by application of tension to the at least one locking element, without requiring knotting thereof.

Preferably, the inserting the flexible sleeve into a channel includes pulling on a pulling suture attached to the flexible sleeve to position the flexible sleeve such that a ring-shaped collar portion thereof lies against an opening of the channel.

In accordance with a preferred embodiment of the present invention the lockably tightening includes threading a loop of one of the at least one lockable suture through a loop defined by a slip noose-like end of one of the at least one suture locking loop element, such that a length of one of the at least one lockable suture, extending between a pair of the series of adjacent mutually spaced suture engagement elements, lies within the loop. Additionally, the lockably tightening also includes tightening the loop defined by the slip noose-like end, thereby locking the length of lockable suture extending between the pair of adjacent mutually spaced suture locking elements by pulling on a length of suture material forming part of one of the at least one suture locking element. Preferably, the pulling on the length of suture locking element also causes a cylindrical portion of the flexible sleeve to be pulled forwardly by the slip noose-like end and to be bunched inside the bone just rearward of an opening of the channel, thereby protecting a cortical layer of the bone against cutting by the suture material.

### BRIEF DESCRIPTION OF THE DRAWINGS

The present invention will be understood and appreciated more fully from the following detailed description, taken in conjunction with the drawings in which:

FIGS. 1A & 1B are simplified pictorial and exploded view illustrations of a suture assembly constructed and operative in accordance with a preferred embodiment of the present invention;

FIGS. 2A and 2B are simplified plan view and sectional view illustrations of part of the suture assembly of FIGS. 1A & 1B taken respectively in a direction along an arrow IIA and along a section line IIB-IIB in FIG. 1A;

FIGS. 3A and 3B are simplified plan view and sectional view illustrations of part of the suture assembly of FIGS. 1A & 1B taken respectively in a direction along an arrow IIIA and along a section line IIIB-IIIB in FIG. 1A;

FIGS. 4A and 4B are simplified plan view and sectional view illustrations of part of the suture assembly of FIGS. 1A & 1B taken respectively in a direction along an arrow IVA and along a section line IVB-IVB in FIG. 1A:

FIGS. 5A, 5B, 5C, 5D, 5E and 5F are simplified pictorial illustrations of typical stages in a suture technique employing the suture assembly of FIGS. 1A-4B in a non-clinical environment; and

FIGS. 6A, 6B, 6C. 6D, 6E, 6F and 6G are simplified pictorial illustrations of typical stages in a suture technique employing the suture assembly of FIGS. 1A-4B in a clinical environment.

### DETAILED DESCRIPTION OF PREFERRED EMBODIMENTS

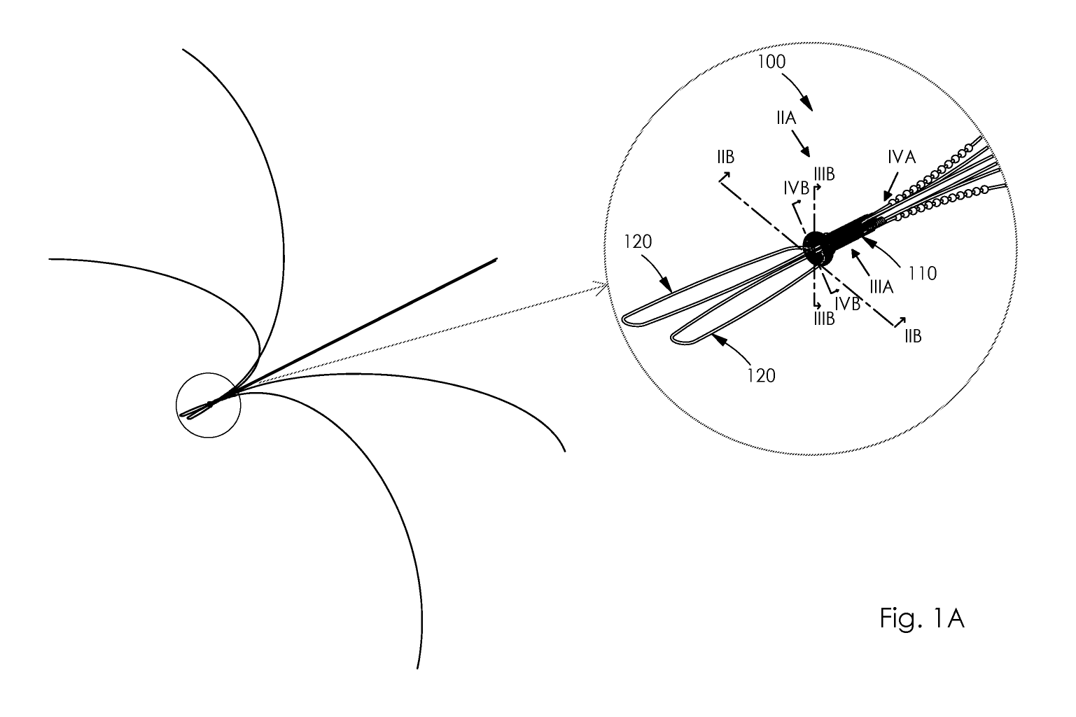

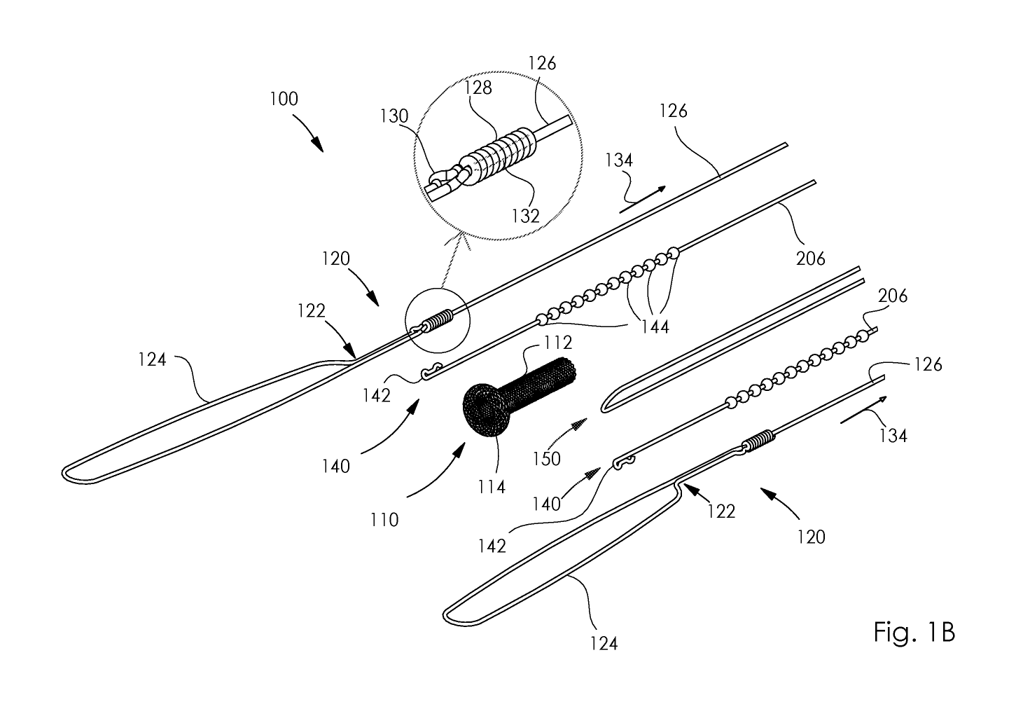

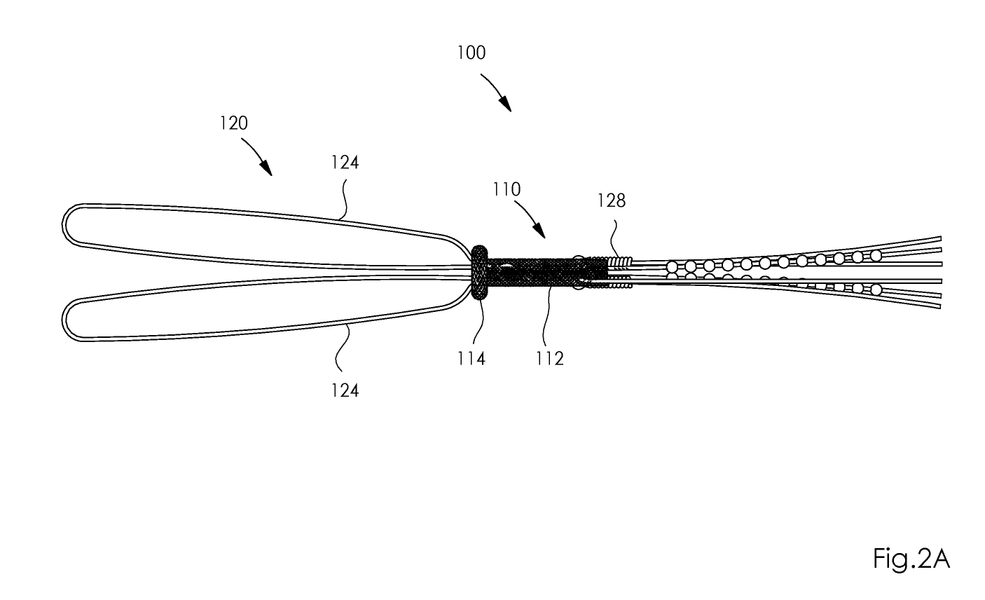

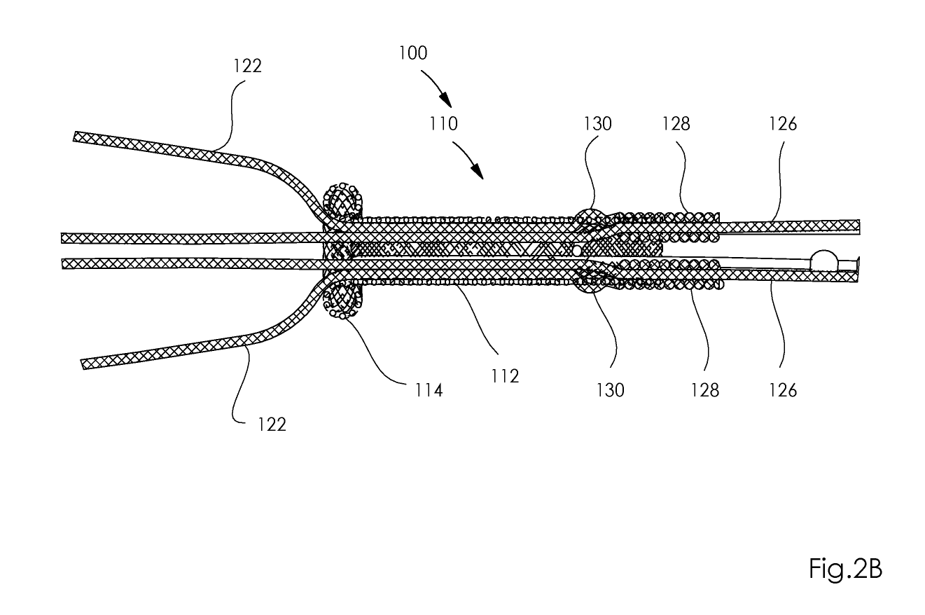

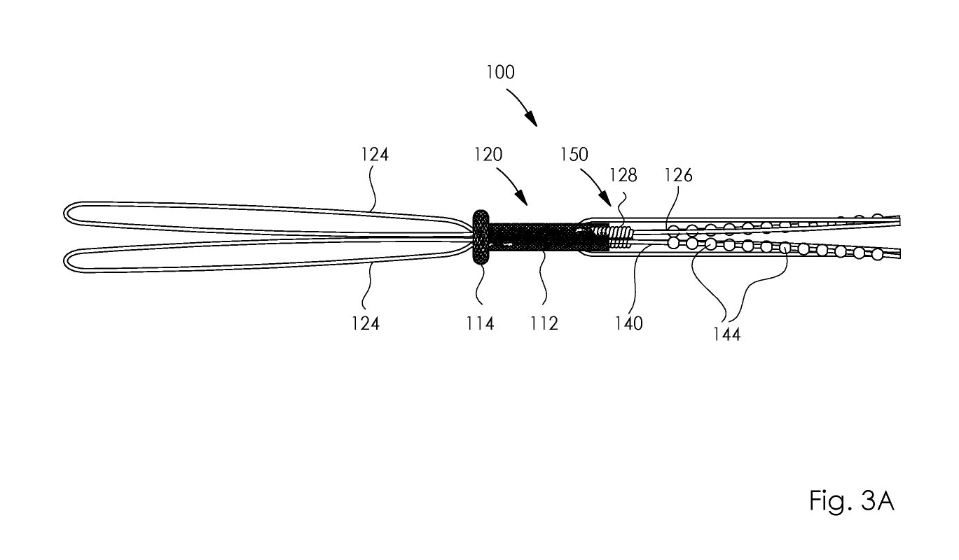

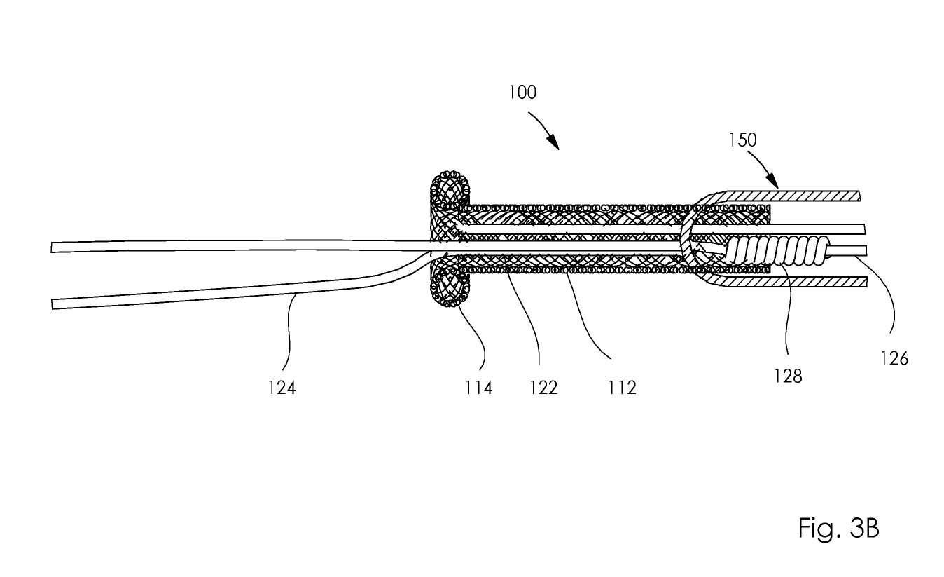

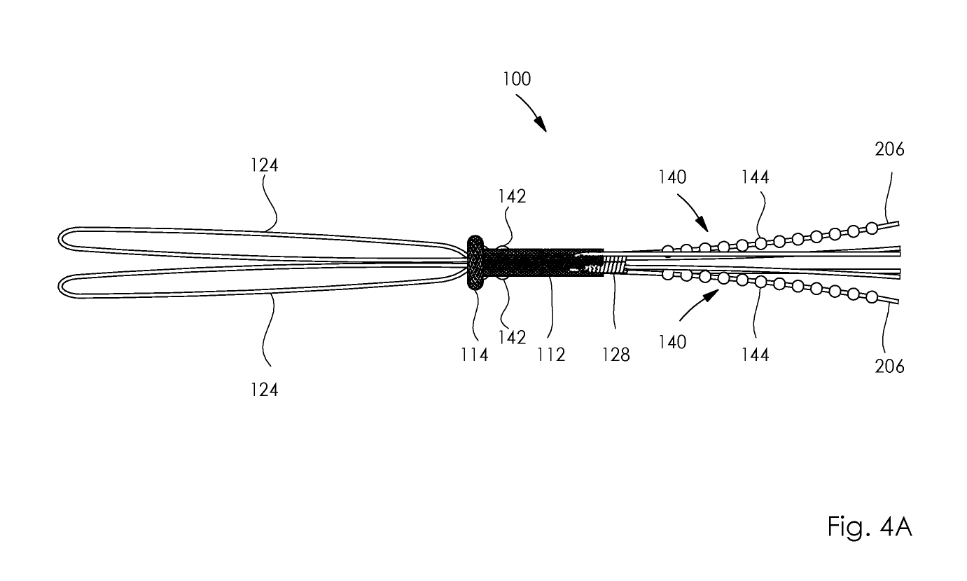

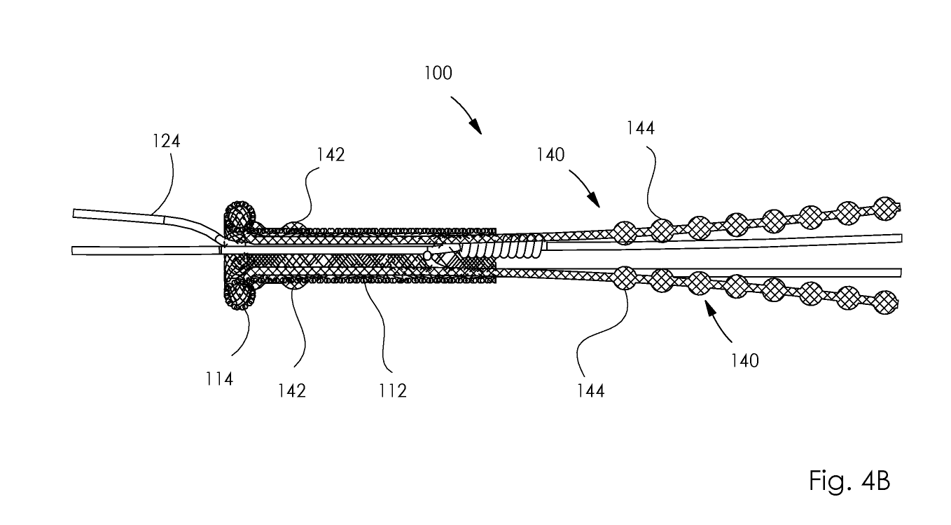

Reference is now made to FIGS. 1A-4B, which are simplified illustrations of a suture assembly 100 constructed and operative in accordance with a preferred embodiment of the present invention. As seen in FIGS. 1A and 1B, suture assembly 100 comprises a flexible sleeve 110, preferably formed of a braided flexible elongate surgical suture material, such as, for example UHMWPE or PET, preferably having a cross-sectional diameter of 0.3 mm. Flexible sleeve 110 preferably comprises a compressible elongate cylindrical portion 112 which is integrally formed at an end thereof with a compressible ring-shaped collar portion 114, having an outer diameter substantially greater than that of cylindrical portion 112. Typically, elongate cylindrical portion 112 has an inner diameter of 2.9 mm and an outer diameter of 3.5 mm, and ring-shaped collar portion 114 has an inner diameter of 2.9 mm and an outer diameter of 7 mm. Flexible sleeve 110 may be formed by any suitable braiding or other technique and may alternatively be formed of a woven, knitted or non-woven web material.

At least one, and preferably first and second, suture locking loop elements 120 extend through the interior of flexible sleeve 110 and are attached thereto. As seen particularly clearly in FIG. 1B, each of first and second suture locking loop elements 120 is formed of a single length of suture material, typically having a cross-sectional diameter of 0.5 mm. Each of first and second suture locking loop elements 120 is preferably formed with a slip noose-like end 122 defining a flexible loop 124, by winding an end portion of a length 126 of suture material in a coil 128 over an intermediate portion of the length 126 of suture material, such that at least a portion 130 of the length 126 of suture material adjacent a free end 132 thereof extends outwardly through a portion of flexible sleeve 110, for anchoring the slip noose-like end 122 to the flexible sleeve 110. Additionally or alternatively, some or all of the windings of coil 128 may extend through portions of flexible sleeve 110. The free end 132 is located interiorly of coil 128. It is appreciated that the operation of slip noose-like end 122 is such that flexible loop 124 can be readily tightened and locked in a tightened operative orientation by pulling on length 126 of suture material in a direction indicated by an arrow 134 relative to coil 128 and flexible sleeve 110. The slip noose-like end 122 locks the length 126 of suture material against retraction and consequent loosening of the engagement of loop 124 with a lockable suture 140 without requiring knotting.

At least one, and preferably first and second, lockable sutures 140 are either mounted onto or integrally formed with flexible sleeve 110. In the illustrated embodiment of FIG. 1B, the first and second lockable sutures 140 are secured to flexible sleeve 110 by an end attachment portion 142. Each of first and second lockable sutures 140 is preferably formed with a series of mutually spaced suture engagement elements 144, typically in the form of beads, fixed thereto and extending along at least a portion thereof, typically rearwardly of flexible sleeve 110.

A pulling suture 150 is preferably looped through flexible sleeve 110, at a location near the rearward end of cylindrical portion 112 thereof, and is used for pulling the flexible sleeve 110 into a channel, such as a transosseous channel in a bone.

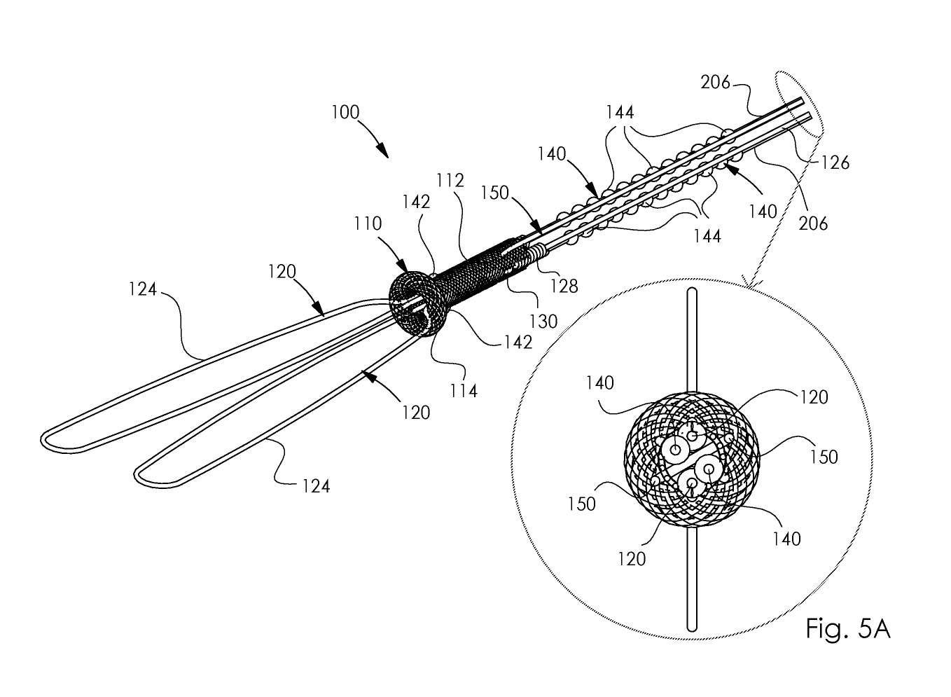

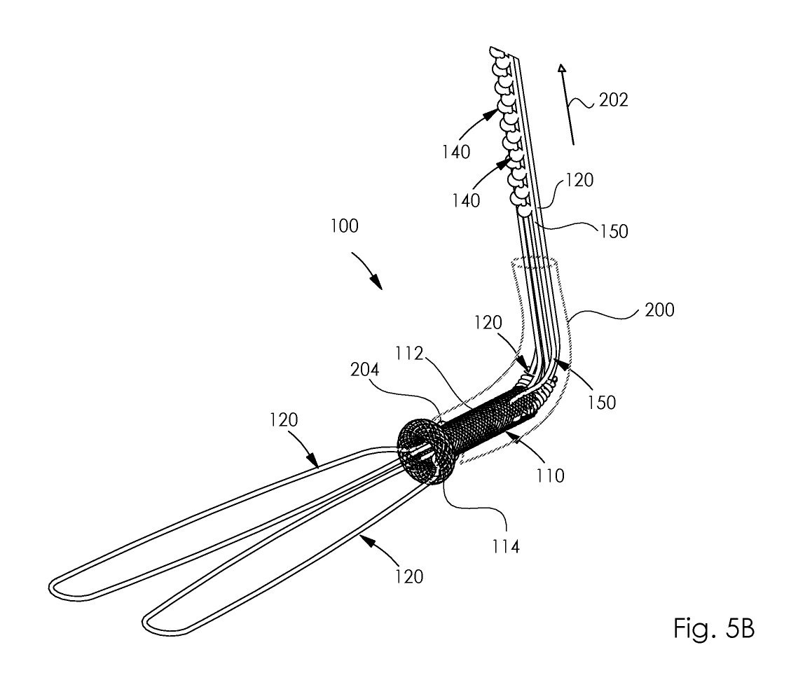

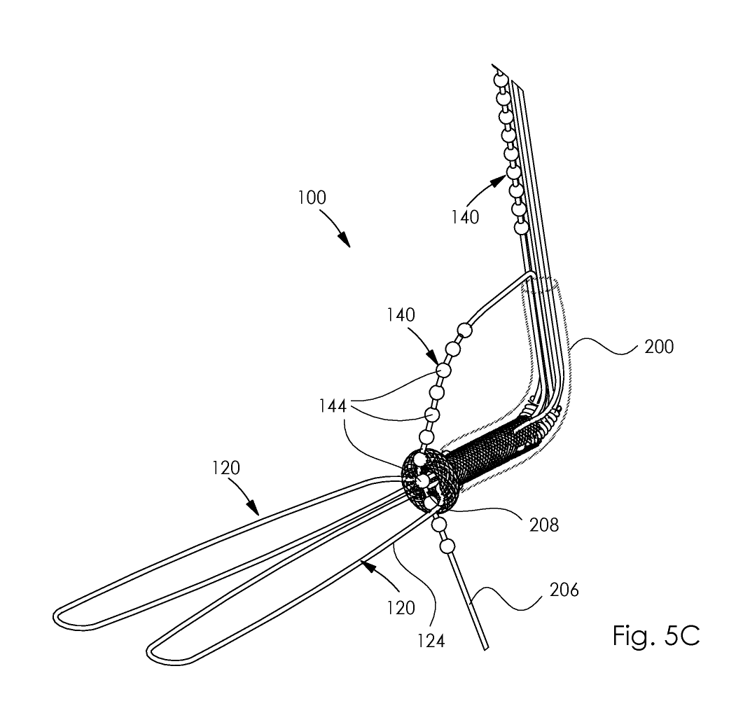

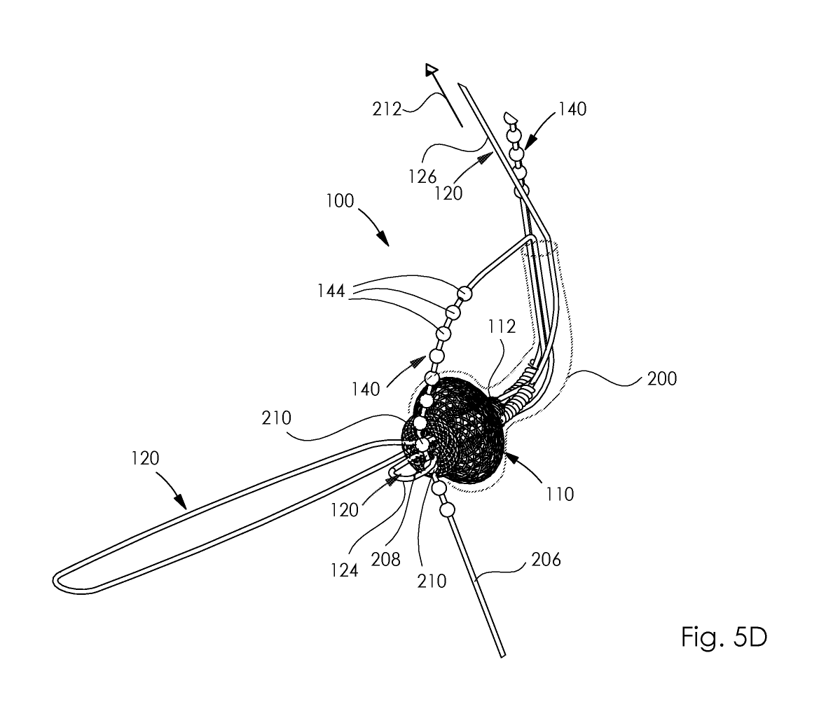

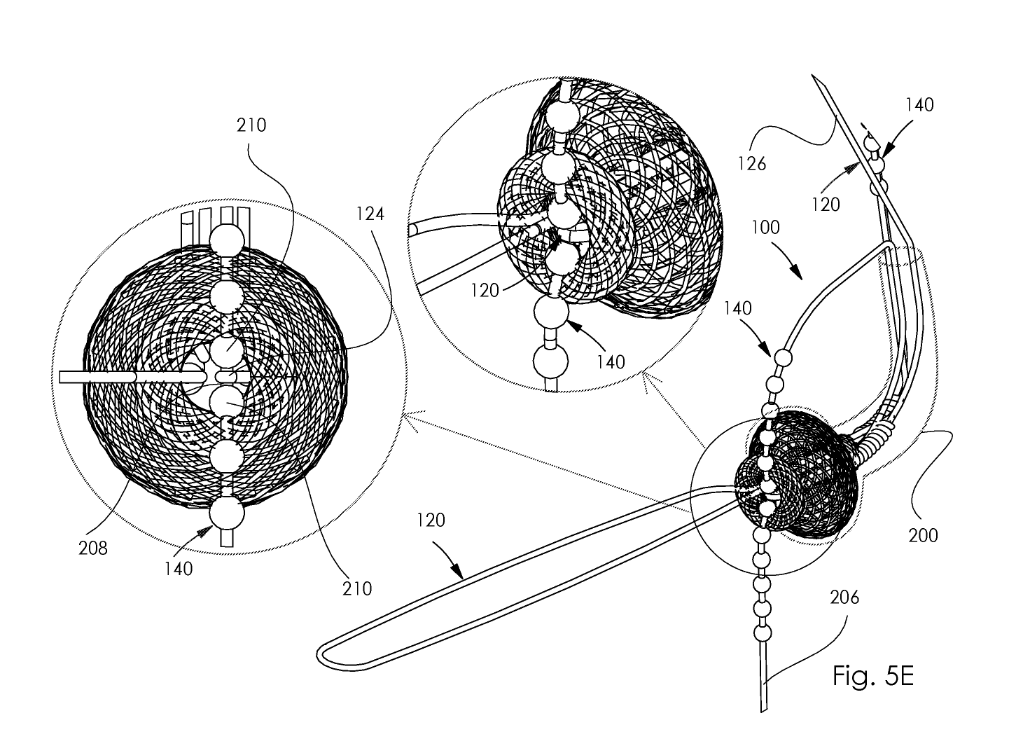

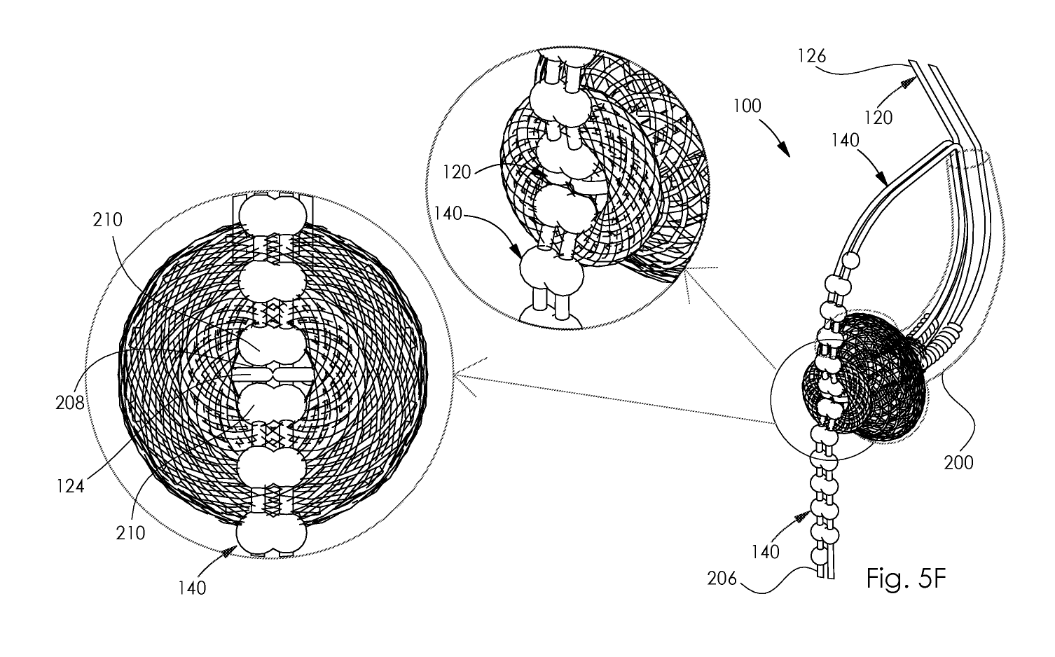

Reference is now made to FIGS. 5A, 5B. 5C, 5D, 5E and 5F, which are simplified pictorial illustrations of typical stages in a suture technique employing the suture assembly of FIGS. 1A-4B in a non-clinical environment.

FIG. 5A illustrates the suture assembly 100 of FIGS. 1A-1B prior to installation thereof. The elements thereof are identified by the same reference numerals employed in the above description of FIGS. 1A-4B.

FIG. 5B shows the suture assembly 100 of FIG. 5A having been pulled rearwardly into a curved channel 200 by pulling on free ends of pulling suture 150 in a direction indicated by an arrow 202. The suture assembly 100 is preferably positioned in channel 200 such that ring-shaped collar portion 114 thereof lies against a forward-facing opening 204 of channel 200.

FIG. 5C shows a free end 206 of a first one of first and second lockable sutures 140 having been threaded through one of loops 124 of a slip noose-like end 122 of one of first and second suture locking loop elements 120, such that a length 208 of a lockable suture 140 extending between a pair of adjacent mutually spaced suture engagement elements 144, typically in the form of beads, lies within the loop 124.

FIG. 5D shows the loop 124 of slip noose-like end 122 being tightened and locked into locking engagement with length 208 of lockable suture 140, extending between a pair of adjacent mutually spaced suture engagement elements 144, here both designated by reference numeral 210, by pulling on length 126 of suture material of suture locking element 120 in a direction indicated by an arrow 212. It is noted that pulling on length 126 of suture locking element 120 in the direction indicated by arrow 212 also causes the cylindrical portion 112 of the flexible sleeve 110 to be pulled forwardly by the slip noose-like end 122 and to be bunched inside curved channel 200 just rearward of forward-facing opening 204 of curved channel 200.

FIG. 5E shows full tightening and locking of length 208 of one lockable suture 140 between adjacent engagement elements 210 by loop 124. FIG. 5F shows full tightening and locking of both lockable sutures 140 by repeating the steps described hereinabove with respect to FIGS. 5C, 5D and 5E for the second lockable suture 140.

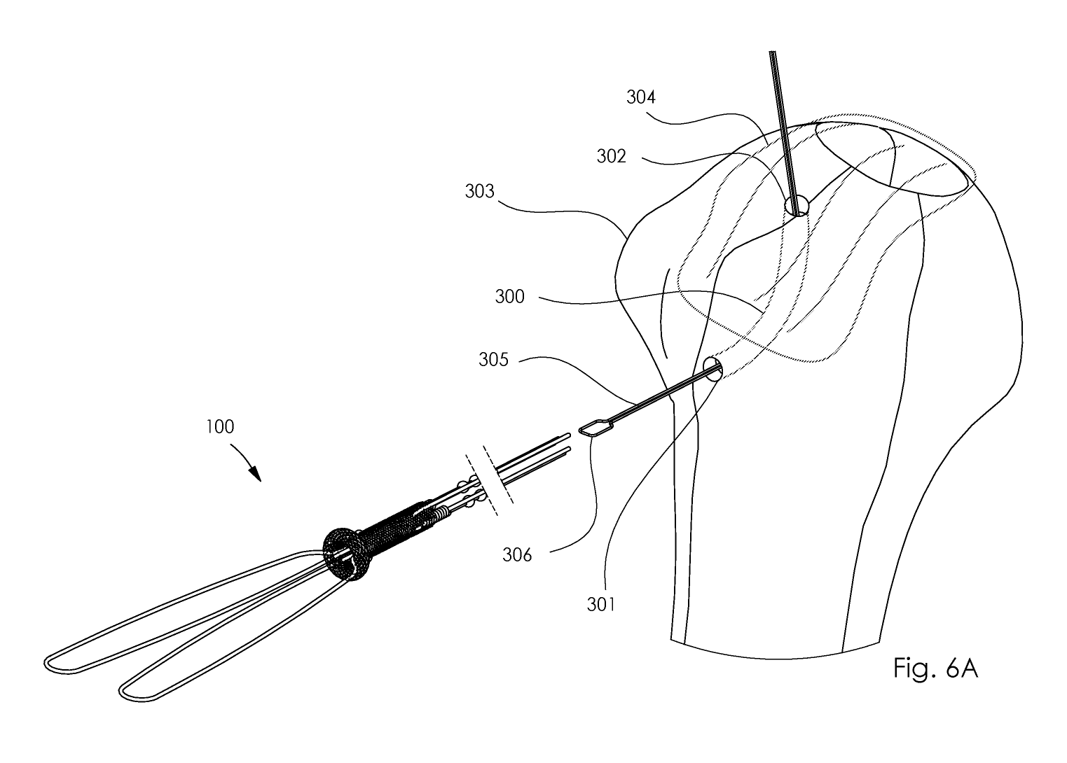

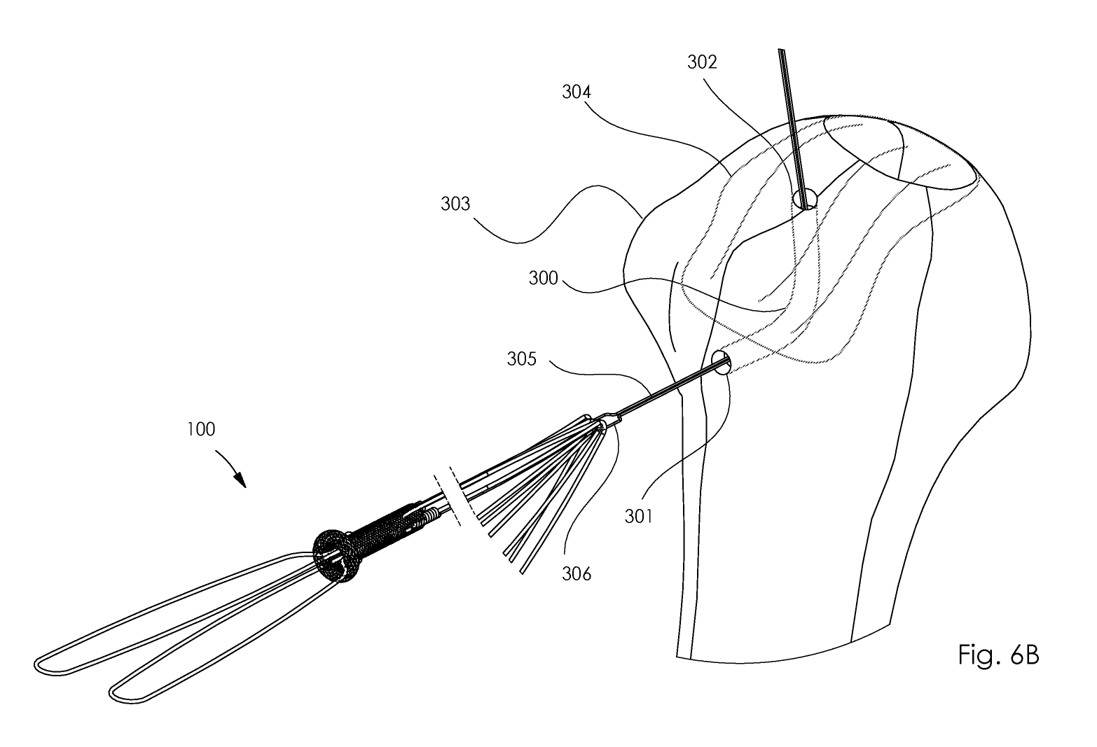

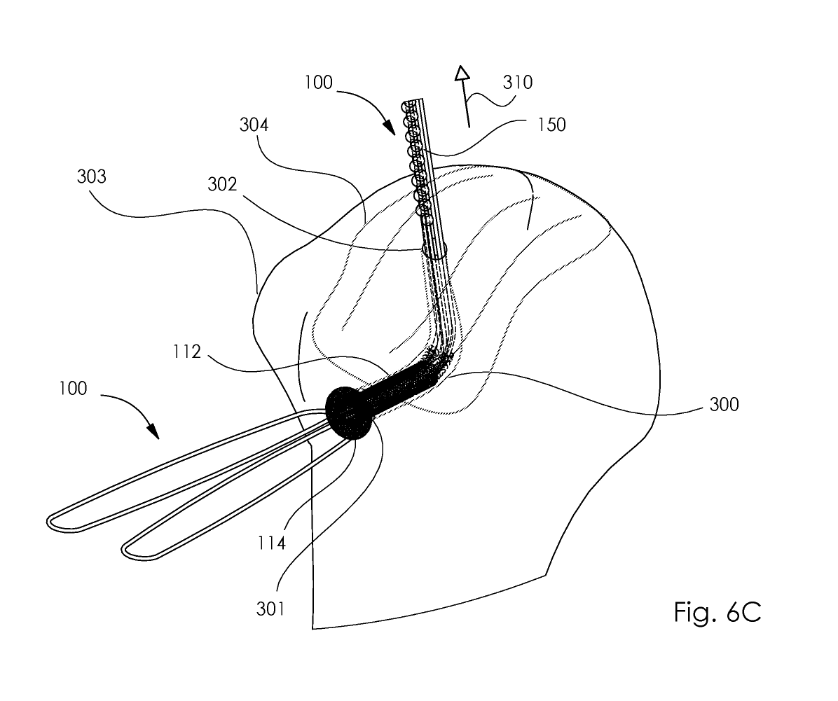

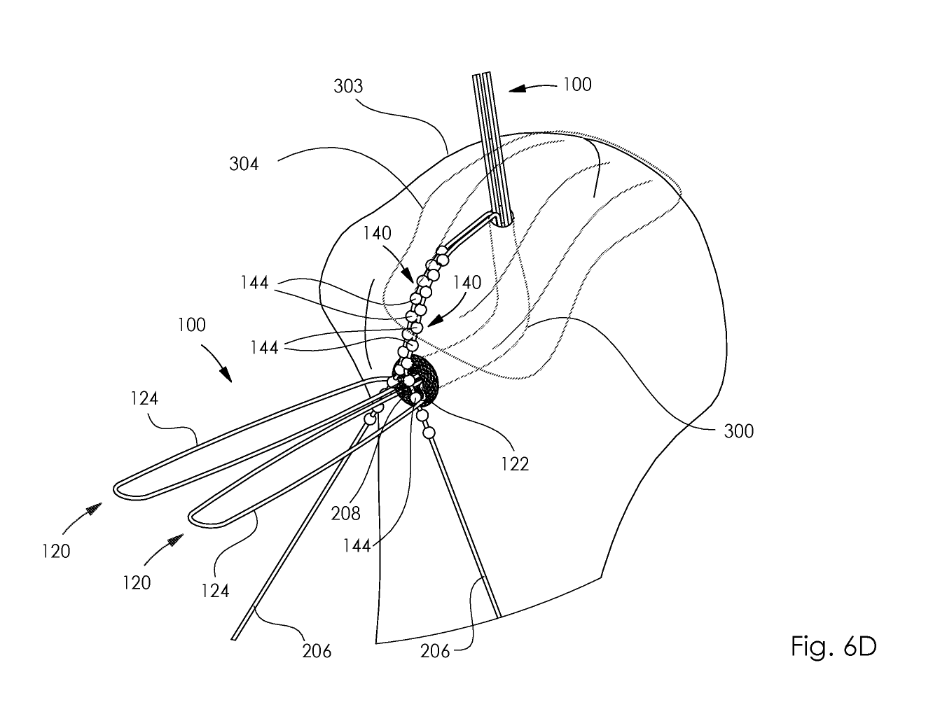

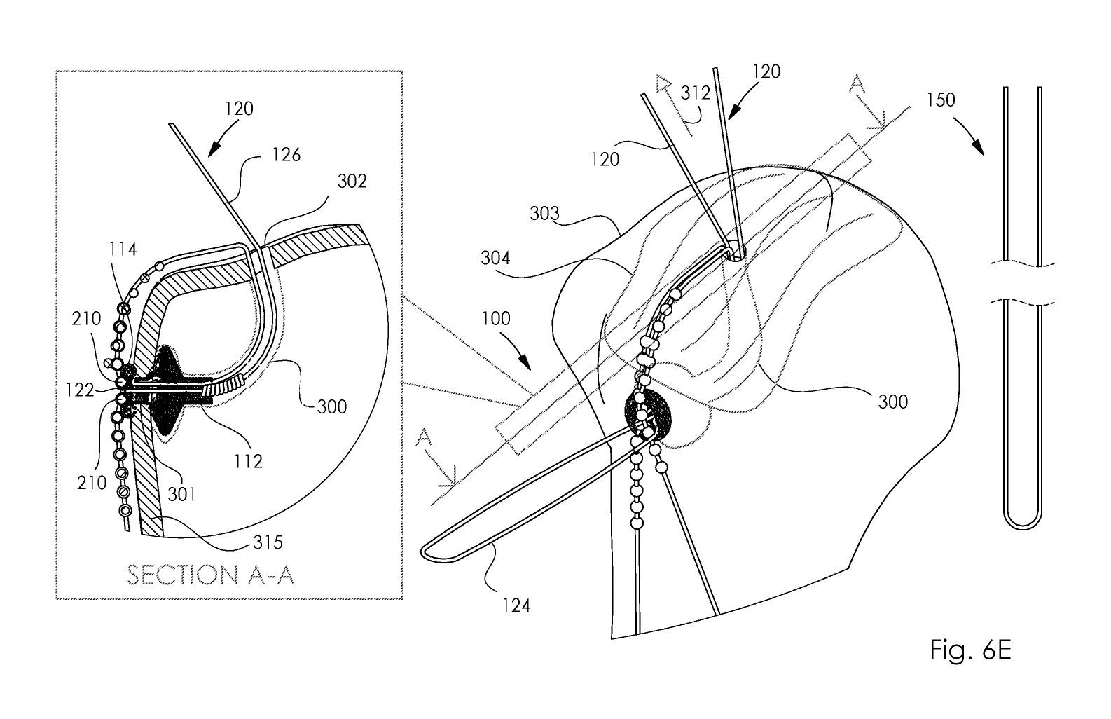

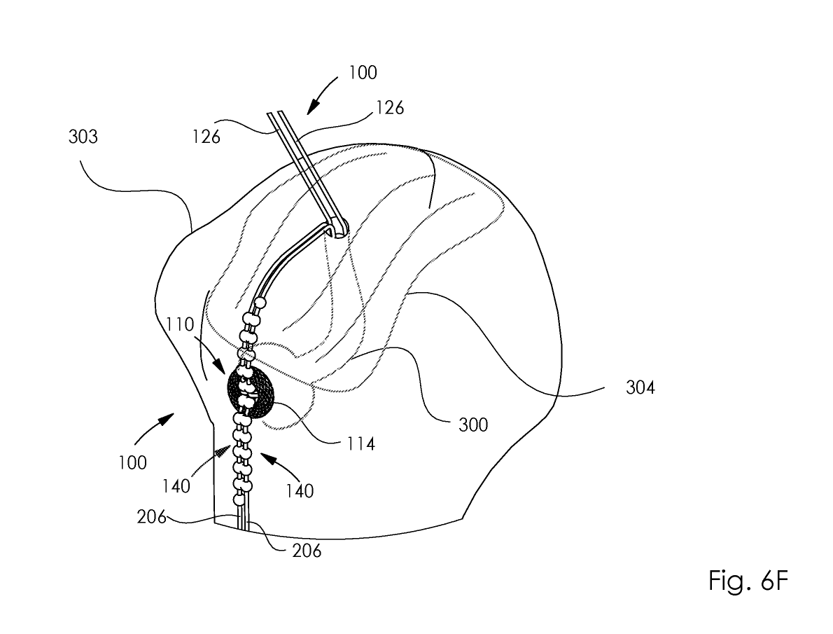

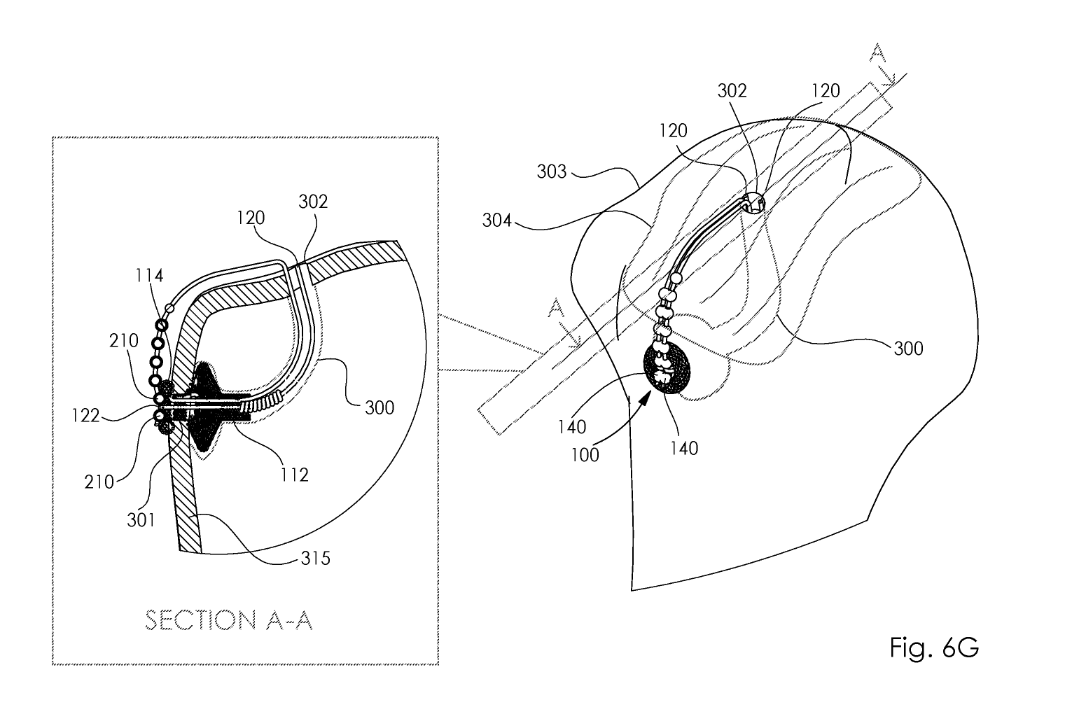

Reference is now made to FIGS. 6A, 6B, 6C, 6D, 6E, 6F, 6G and 6H, which are simplified pictorial illustrations of typical stages in a suture technique employing the suture assembly of FIGS. 1A-4B in a clinical environment.

As seen in FIG. 6A, as an initial step, prior to insertion of the suture assembly 100, a transosseous channel 300, having a forward-facing opening 301 and a top-facing opening 302, is formed in a bone 303, having a ligament 304 extending thereover, and a snare wire 305 or suture defining a loop 306 is threaded through the transosseous channel 300. This may be accomplished by any suitable technique, such as for example using the teachings of any of the patent publications referenced hereinabove, the disclosures of which are hereby incorporated by reference.

As seen in FIG. 6B, the suture assembly 100, described above with reference to FIGS. 1A-5F is threaded through loop 306, preferably as shown in FIG. 6B. The snare wire 305 is then pulled back through the transosseous channel 300, preferably until the suture assembly 100 is engaged in the transosseous channel 300.

As seen in FIG. 6C, the suture assembly 100 of FIG. 6B is pulled rearwardly into channel 300 by pulling on free ends of pulling suture 150 in a direction indicated by an arrow 310. The suture assembly 100 is preferably positioned in channel 300 such that ring-shaped collar portion 114 thereof lies against forward-facing opening 301 of channel 300.

FIG. 6D shows free ends 206 of first and second lockable sutures 140 having been threaded through respective loops 124 of slip noose-like ends 122 of respective first and second suture locking loop elements 120, such that a length 208 of each of first and second lockable sutures 140, extending between a pair of adjacent mutually spaced suture engagement elements 144, typically in the form of beads, lies within each loop 124.

FIG. 6E shows the loop 124 of slip noose-like end 122 being tightened into locking engagement with length 208 of lockable suture 140 extending between a pair of adjacent mutually spaced suture locking engagement elements 210, by pulling on length 126 of suture material of suture locking element 120 in a direction indicated by an arrow 312. It is noted that pulling on length 126 of suture locking element 120 in a direction indicated by arrow 312 also causes the cylindrical portion 112 of the flexible sleeve 110 to be pulled forwardly by the slip noose-like end 122 and to be bunched just rearwardly of the cortical layer 315 of the bone 303 surrounding the forward-facing opening 304 of channel 300, thus expanding the channel 300 thereat. It is noted that pulling suture 150 is preferably disengaged from flexible sleeve 110 at this stage.

It is also noted that the cortical layer 315 surrounding channel 300 at forward-facing opening 301 is protected against cutting by the sutures both by compressible ring-shaped collar portion 114, which lies outside of forward-facing opening 301, and by the compressed cylindrical portion 112 of the flexible sleeve 110, which lies within an expanded portion of channel 301 just inside of the cortical layer 315.

FIG. 6F shows full tightening and locking of both lockable sutures 140, by repeating the steps described hereinabove with respect to FIG. 6E with the second lockable suture 140 and second loop 124, without requiring knotting of the lockable sutures 140.

FIG. 6G shows lockable sutures 140 extended over ligament 304 and securing it to bone 303 between forward-facing opening 301 and top-facing opening 302 of channel 300. The free ends of lockable sutures 140 and of all other sutures forming part of the suture assembly 100 are cut so as not to extend outwardly from forward-facing opening 301 and top-facing opening 302. Lockable sutures 140 remain locked in place by respective suture locking elements 120 within flexible sleeve 110, which are locked in their tightened operative orientation by the operation of slip noose-like ends 122.

It will be appreciated by persons skilled in the art that the present invention is not limited to what has been particularly shown and described hereinabove. Rather the scope of the present invention includes both combinations and subcombinations of various features as well as modifications thereof which are not known in the art.

1. A bone suture assembly comprising:
a flexible sleeve;
at least one lockable suture associated with said flexible sleeve and having a series of mutually spaced suture engagement elements extending along at least a portion thereof; and
at least one suture locking element defining a loop which can be lockably tightened in locking engagement with a corresponding one of said at least one lockable suture by application of tension to said at least one suture locking element, without requiring knotting thereof.

2. A bone suture assembly according to claim 1 and wherein said at least one suture locking element comprises an elongate flexible element having first and second ends.

3. A bone suture assembly according to claim 2 and wherein said at least one suture locking element is configured to be lockably tightened by pulling on said first end of said elongate flexible element.

4. A bone suture assembly according to claim 1 and wherein said at least suture one locking element defines a loop which can be lockably tightened in locking engagement with a corresponding one of said at least one lockable suture at a location therealong intermediate two of said series of mutually spaced suture engagement elements.

5. A bone suture assembly according to claim 1 and wherein said flexible sleeve is a generally cylindrical sleeve formed of suture material.

6. A bone suture assembly according to claim 1 and wherein said at least one suture locking element is formed of suture material.

7. A bone suture assembly according to claim 1 and wherein said flexible sleeve is braided from surgical suture material.

8. A bone suture assembly according to claim 1 and wherein said flexible sleeve comprises a compressible elongate cylindrical portion, which is integrally formed at an end thereof with a compressible ring-shaped collar portion.

9. A bone suture assembly according to claim 1 and wherein said at least one suture locking element comprises first and second suture locking loop elements, which extend through an interior of said flexible sleeve and are attached thereto.

10. A bone suture assembly according to claim 1 and wherein each of said at least one suture locking element comprises a length of suture material having a slip noose-like end defining said loop by winding an end portion of said length of suture material in a coil over an intermediate portion of said length of suture material.

11. A bone suture assembly according to claim 10 and wherein a portion of said length of suture material adjacent a free end thereof extends outwardly through a portion of said flexible sleeve for anchoring said slip noose-like end to said flexible sleeve.

12. A bone suture assembly according to claim 10 and wherein at least one winding of said coil extends through a portion of said flexible sleeve.

13. A bone suture assembly according to claim 10 and wherein operation of said slip noose-like end is such that said flexible loop can be readily tightened and locked in a tightened operative orientation by pulling on said length of suture material in a given direction relative to said coil and said flexible sleeve, whereby said slip noose-like end locks said length of suture material against retraction and consequent loosening of said locking engagement of said loop with said lockable suture without requiring knotting.

14. A bone suture assembly according to claim 1 and wherein said series of mutually spaced suture engagement elements are in fixed positions along each of said at least one lockable suture.

15. A bone suture assembly according to claim 1 and also comprising at least one pulling suture looped through flexible sleeve and suitable for pulling the flexible sleeve into a transosseous channel in a bone.

16. A method of retaining a suture onto a bone without requiring knotting comprising:
providing a bone suture assembly comprising a flexible sleeve, at least one lockable suture, extending through said flexible sleeve and having a series of mutually spaced suture engagement elements extending along at least a portion thereof, and at least one suture locking element defining a loop;
inserting said flexible sleeve into a channel extending through said bone; and
lockably tightening said at least one locking element in locking engagement with said at least one lockable suture by application of tension to said at least one locking element, without requiring knotting thereof.

17. A method of retaining a suture onto a bone without requiring knotting according to claim 16 and wherein said inserting said flexible sleeve into a channel comprises pulling on a pulling suture attached to said flexible sleeve to position said flexible sleeve such that a ring-shaped collar portion thereof lies against an opening of said channel.

18. A method of retaining a suture onto a bone without requiring knotting according to claim 16 and wherein said lockably tightening comprises:
threading a loop of one of said at least one lockable suture through a loop defined by a slip noose-like end of one of said at least one suture locking loop element, such that a length of one of said at least one lockable suture, extending between a pair of said series of adjacent mutually spaced suture engagement elements, lies within said loop.

19. A method of retaining a suture onto a bone without requiring knotting according to claim 18 and wherein said lockably tightening also comprises:
tightening said loop defined by said slip noose-like end, thereby locking said length of lockable suture extending between said pair of adjacent mutually spaced suture locking elements by pulling on a length of suture material forming part of one of said at least one suture locking element.

20. A method of retaining a suture onto a bone without requiring knotting according to claim 19 and wherein said pulling on said length of suture locking element also causes a cylindrical portion of said flexible sleeve to be pulled forwardly by said slip noose-like end and to be bunched inside the bone just rearward of an opening of said channel, thereby protecting a cortical layer of said bone against cutting by said suture material.
